(19) United States
(12) Patent Application Publication
(10) Pub. No.: US 2023/0404437 A1
(43) Pub. Date: Dec. 21, 2023

(54) SYSTEM AND METHOD FOR ASSESSING NEUROMUSCULAR DISEASE

(71) Applicant: MOVEMED LIMITED, Cambridge (GB)

(72) Inventor: Benjamin Davies, Histon Cambridgeshire (GB)

(73) Assignee: MOVEMED LIMITED, Cambridge (GB)

(21) Appl. No.: 18/037,989

(22) PCT Filed: Nov. 19, 2021
(86) PCT No.: PCT/GB2021/053003

§ 371 Date: May 19, 2023

(30) Foreign Application Priority Data
Nov. 20, 2020 (GB) 2018262.2

(51) Int. Cl. A61B 5/11; A61B 5/00

(52) U.S. Cl. CPC A61B 5/1124; A61B 5/4041; A61B 5/4519; A61B 5/748; A61B 5/4842

(57) ABSTRACT

A method of assessing neuromuscular disease in a subject, the method comprising: recording the subject performing one or more tasks on a device, wherein the one or more tasks requires the subject to interact with the device by touching the device, including recording touch interactions with the device using one or more sensors of the device; determining one or more parameters from the recorded touch interactions; comparing the determined parameters with predetermined parameters.

### TECHNICAL FIELD

The present invention relates to methods of assessing neuromuscular disease in a subject.

### BACKGROUND ART

It is estimated 20% of the human population will suffer neuromuscular disease in their lifetime. Care and research for neuromuscular disease generally relies on subjective, qualitative assessments measured at specific timepoints and typically during time-limited hospital consultations. This can limit the effectiveness of treatment and increase significantly clinical trial cost and duration.

Degenerative Cervical Myelopathy (DCM) is one example of such a neuromuscular disease. In DCM, a spinal cord injury caused by arthritis affecting 2% of adults, poor assessments contribute to delayed (70%) and missed (50%) diagnosis, reducing treatment response.

Moreover, Phase III medicine trials are decided by outcomes such as the modified Japanese Orthopaedic Association Score (mJOA); an ordinal categorisation of disabilities e.g. “Unable to button a shirt but can eat with a spoon” (2 points) vs. “Able to button shirt with great difficulty” (3 points). This assessment is subjective.

Embodiments of the present invention aim to at least partially address problems identified above, for example by providing one or more of: objective data, data obtained remotely, data obtained more frequently, data obtained during routine daily activity and data obtained with minimal burden on a subject or an assessor.

### SUMMARY OF THE INVENTION

According to a first aspect of the invention, there is provided a method of assessing neuromuscular disease in a subject, the method comprising: recording the subject performing one or more tasks on a device, wherein the one or more tasks requires the subject to interact with the device by touching the device, including recording touch interactions with the device using one or more sensors of the device; determining one or more parameters from the recorded touch interactions; comparing the determined parameters with predetermined parameters.

Optionally, the one or more tasks includes a task presented to the subject via a user interface of the device.

Optionally, the one or more tasks includes a task performed during alternative use of the device, not a task presented to the subject via a user interface of the device specifically for assessing neuromuscular disease.

Optionally, the task includes repeatedly touching single a target.

Optionally, the task includes touching a plurality of different targets.

Optionally, the task includes typing a character string.

Optionally, the one or more parameters include indicators of interaction speed. Optionally, the one or more parameters include: a rate of touch interactions, variation in the rate of the touch interactions over time, and time taken for the subject to complete the task.

Optionally, the one or more parameters include indicators of interaction accuracy. Optionally, the one or more parameters include: accuracy of the touch interactions, variation in the accuracy of the touch interactions over time, total number of errors by the subject, rate of errors by the subject, time for the subject to make a predetermined number of errors.

According to a second aspect of the invention there is provided a method of assessing neuromuscular disease in a subject, the method comprising: recording the subject performing one or more tasks on a device, wherein the one or more tasks requires the subject to manipulate the device, including recording movement of the device using one or more sensors of the device; determining one or more parameters from the recorded movement of the device; comparing the determined parameters with predetermined parameters.

Optionally, the one or more tasks includes a task presented to the subject via a user interface of the device.

Optionally, the one or more tasks includes a task performed during alternative use of the device, not a task presented to the subject via a user interface of the device specifically for assessing neuromuscular disease.

Optionally, the task includes holding the device in a predefined way for a predefined duration.

Optionally, the one or more parameters include: positional deviation from a predetermined position over time, rotational deviation from a predetermined orientation over time, oscillation frequency, variation in oscillation frequency over time, oscillation magnitude, and variation in oscillation magnitude over time.

Optionally, the task includes moving the device in a predefined way. Optionally, the task includes walking with the device.

Optionally, the one or more parameters include: stride frequency, variation in stride frequency over time, stride length, variation in stride length over time, stride width, variation in stride width over time.

According to a third aspect of the invention, there is provided a method of assessing neuromuscular disease in a subject, the method comprising the steps of the method of the first aspect and the steps of the method of the second aspect.

In the method of any preceding aspect, optionally the at least one test is performed bilaterally; and the one or more determined parameters includes differences between parameters for each side.

In the method of any preceding aspect, optionally the predetermined parameters include one or more of: a baseline for the subject, a baseline for a predetermined population group, and a baseline for a predetermined clinical group.

In the method of any preceding aspect, optionally the method further comprises determining an assessment score based on the comparison between the determined parameters and the predetermined parameters, wherein the assessment score indicates the severity of neurological disease in the subject.

In the method of any preceding aspect, optionally the method further comprises diagnosing the disease and, optionally treating the disease.

In the method of any preceding aspect, optionally the neuromuscular disease is myelopathy, optionally, degenerative cervical myelopathy.

In the method of any preceding aspect, optionally the device is a handheld or wearable device, such as a smart phone, a tablet computer or a smart watch.

According to a fourth aspect of the invention there is provided a computer program that when executed on a handheld or wearable device, such as a smart phone, a tablet computer or a smart watch, is configured to perform the method of any one of the previous aspects.

According to a fifth aspect of the invention there is provided a handheld or wearable device, such as a smart phone, a tablet computer or a smart watch, loaded with a computer program that when executed on a handheld or wearable device is configured to perform the method of any one of the previous aspects.

According to a sixth aspect of the invention there is provided a system for assessing neuromuscular disease in a subject, the system comprising: a device comprising a touch screen and one or more sensors, wherein the touch screen is configured to present one or more tasks to the subject and record the subject performing the one or more tasks, wherein the one or more tasks requires the subject to interact with the device by touching the device, and the one or more sensors are configured to record touch interactions with the device; one or more processors configured to determine one or more parameters from the recorded touch interactions and compare the determined parameters with predetermined parameters.

According to a seventh aspect of the invention there is provided a system for assessing neuromuscular disease in a subject, the system comprising: a device comprising one or more sensors, wherein the device is configured to present one or more tasks to the subject and record the subject performing one or more tasks, wherein the one or more tasks requires the subject to manipulate the device, and the one or more sensors are configured to record movement of the device; one or more processors configured to determine one or more parameters from the recorded movement of the device and compare the determined parameters with predetermined parameters.

Optionally, the one or more processors is configured to determine an assessment score based on the comparison between the determined parameters and the predetermined parameters, wherein the assessment score indicates the severity of neurological disease in the subject.

### BRIEF DESCRIPTION OF THE DRAWINGS

Further features of the invention will be described below with reference to the accompanying drawings in which:

FIG. 1 shows an example task;

FIG. 2 shows another example task;

FIG. 3 shows another example task;

FIG. 4 shows another example task;

FIG. 5 shows another example task;

FIG. 6 shows an example pain assessment;

FIG. 7A shows the average total number of taps for the test of FIG. 1;

FIG. 7B shows the difference in time between taps at the start of the test of FIG. 1 and the end of the test;

FIG. 8A shows the average typing speed of subjects during the test of FIG. 3 vs mJOA score;

FIG. 8B shows the number of mistakes made vs mJOA score for the test of FIG. 3;

FIG. 9A shows the positional displacement of the device during the test of FIG. 4;

FIG. 9B shows the difference in average oscillation frequency in the y-axis between fourth and first quartiles of the test of FIG. 4 vs mJOA score;

FIG. 10 shows the number of taps over the duration of the test for dominant and non-dominant hands;

FIG. 11 shows tapping accuracy over the duration of the test for dominant and non-dominant hands;

FIG. 12 shows bilateral difference in the number of taps over several weeks of testing;

FIG. 13 shows bilateral difference in tapping accuracy over several weeks of testing;

FIG. 14 shows acceleration for dominant and non-dominant hands;

FIG. 15 shows angular speed for dominant and non-dominant hands;

FIG. 16 show bilateral differences for acceleration over several weeks of testing;

FIG. 17 show bilateral differences for angular speed over several weeks of testing;

FIG. 18 shows angular speed for the holding phase and the walking phase;

FIG. 19 shows acceleration for the holding phase and the walking phase;

FIG. 20 shows angular speed over several weeks of testing;

FIG. 21 shows acceleration over several weeks of testing.

### DETAILED DESCRIPTION

Neuromuscular disease is estimated to affect 20% of adults in their lifetime. The present invention provides methods for assessing neuromuscular disease. In the context of the present disclosure, the term neuromuscular disease may refer to diseases significantly affecting motor and/or sensory function through injury of the nervous system, and/or the muscular system and/or the skeletal articulating joints. Nervous system disease may therefore include the spinal cord (myelopathy), nerve roots exiting the spinal cord (radiculopathy), or nerve branches between the exiting nerve root and their termination (neuropathy). Skeletal articulating joints may therefore include the hip, knee, fingers, wrist, or joints of the spine. DCM is a primary example of a specific neuromuscular disease to which the present disclosure may be applied. Examples of diseases to which the present disclosure may be applied include most preferably: DCM, Degenerative Cervical Radiculopathy, Carpal Tunnel Syndrome, Lumbar Canal Stenosis, Chronic Subdural Haematoma, Metastatic Spinal Cord Compression, Inflammatory Arthritis of the hand (e.g. Rheumatoid or Psoriatic), and Normal Pressure Hydrocephalus. Second most preferably: Hereditary Spastic Paraplegia (HSP), Amyotrophic Lateral Sclerosis (ALS), Primary Neoplasm of Spinal Cord or Dura (E.g. Ependymoma, Astrocytoma, Meningioma), HIV Associated Myelitis and Myelopathy, Cubital Tunnel Syndrome, and Radial Tunnel Syndrome. Third most preferably: HTLV-1 or HTLV-1 Associated Myelopathy, Stiff Person's Syndrome, Syringomyelia, Lumbar Sciatica, Lumbar Radiculopathy, Dystonia of the Hand (e.g. Writer's Cramp), Myasthenia Gravis, Lambert Eaton Syndrome, and Arnold Chiari Syndromes. Fourth most preferably: Whiplash, Dupuytren's Contracture, and Steroid Induced Myopathy.

The present disclosure provides a number of tests for assessing neuromuscular disease in a subject, including tests for assessing the motor and/or sensory function of a subject. Assessing neuromuscular disease may refer to measuring the severity of symptoms or change in severity of symptoms, such as motor and/or sensory function. Tests of the present disclosure may include active tests and passive tests. Active tests may be tests of a type where a subject is aware that they are being tested. For example, where a test involves the subject performing one or more tasks, an active test may include a task presented to the subject via a user interface of a device.

On the other hand, passive tests may be tests of a type where a subject is not aware that they are being tested, or where they are being tested in the background while doing something else. For example, where a test involves the subject performing one or more tasks, a passive test may include a task performed during alternative use of a device, not a task presented to the subject via a user interface of the device specifically for assessing neuromuscular disease.

Tests of the present disclosure generally comprise recording a subject performing one or more tasks on a device. The tests are preferably performed using portable and/or handheld devices. For example, the tests may be performed using a device such as a smart phone, a tablet computer or a smart watch. Such devices typically include touch sensors, such as a touch screen, and movement sensors, such as gyros and accelerometers. These sensors can be used to record the subject performing one or more tasks.

Additional functionality of such devices, including screens, speakers, and wireless data transfer may also be utilised. For example, screens and/or speakers may be used to communicate information to a subject, such are presenting a task during active testing. Data relating to the tasks, whether raw data or processed data, may be transferred to remote devices and/or servers.

It should be understood that embodiments of the invention may be performed on other devices with the necessary hardware.

Tests of a first type according to the present disclosure may include tasks requiring the subject to interact with the device by touching the device. Touch interactions with the device may be recorded using one or more sensors of the device, e.g. a touch screen. Tests of the first type may be configured to test relatively fine sensorimotor function relating to the movement of a finger or fingers of the subject.

From a clinical standpoint, important characteristics of the subject's fine movement, include speed, accuracy, the effects of fatigue on these characteristics, the consistency of movement and differences between the left and right hands. These characteristics can provide information regarding the subject's sensory-motor function. Therefore, methods according to the present disclosure include determining one or more parameters from the recorded touch interactions. These parameters may include indicators of interaction speed and/or indicators of interaction accuracy.

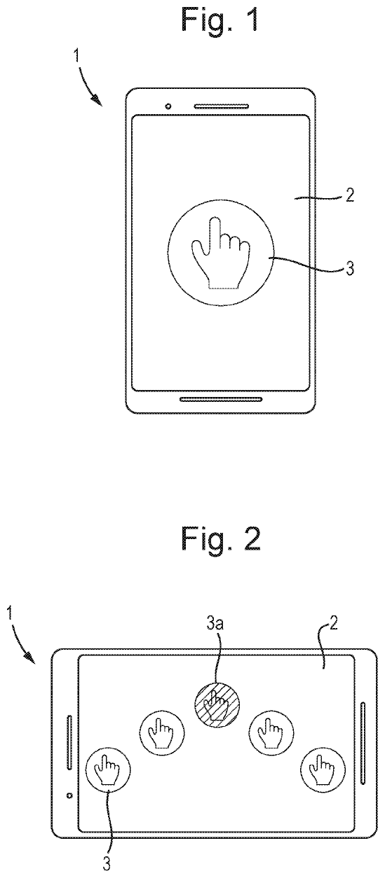

One example of a specific task according to the present disclosure includes repeatedly touching single a target. As shown in FIG. 1, the subject may be instructed to touch the target 3 as many times as possible within a predetermined time-period. The target 3 may be in the form of a button on a touch screen 2 of a device 1.

The time and location (e.g. relative to the target) of each touch interaction may be recorded by the device. Parameters relating to speed determined from the recorded data may include a rate of touch interactions, e.g. total number of touches in a given period. Variation in the rate of the touch interactions over time may be determined, e.g. differences in the rate (e.g. average rate) of touches for different periods (e.g. quartiles of the test duration). Parameters relating to accuracy determined from the recorded data may include the accuracy of the touch interactions, such as distance from a target centre. Variation in the accuracy of the touch interactions over time may be determined.

Another example of a specific task according to the present disclosure includes touching a plurality of different targets. Accordingly, the subject is required to touch different locations. As shown in FIG. 2, the targets 3 may be in the form of a buttons on the touch screen 2 of the device 1. The targets 3 may be presented to the subject concurrently (as shown in FIG. 2), individually or in sub-groups. If the targets are presented concurrently, the subject may be instructed to touch the targets in an order defined prior to performing the task (e.g. left to right) or an order defined during the task, e.g. by indicating a first target and then each subsequent target or simultaneously, using a combination of fingers. This may be indicated visually, e.g. by colour, flashing, etc. (as indicated by the shaded target 3a shown in FIG. 2). If targets are presented individually, the location of each target may be varied, systematically (e.g. left to right), randomly or pseudo-randomly (i.e. predetermined but to give the impression of a random distribution). If the targets are presented in groups, a combination of the above may be utilised, e.g. each group of targets behaving as in the individually presented case and each target within a group behaving like the altogether presented case.

The time (e.g. relative to each target) and location (e.g. relative to each target) of each touch interaction may be recorded by the device. Parameters relating to speed determined from the recorded data may include a rate of touch interactions, e.g. total number of touches in a given period. Variation in the rate of the touch interactions over time may be determined, e.g. differences in the rate (e.g. average rate) of touches for different periods (e.g. quartiles of the test duration). The time taken for the subject to complete the task may also be determined. Parameters relating to accuracy determined from the recorded data may include: accuracy of the touch interactions, such as distance from a target centre. Variation in the accuracy of the touch interactions over time may be determined. The total number of errors by the subject may be determined. For this example, as error might include missing a target, deviating from the centre of a target by a predefined amount, or touching an incorrect target. The rate of errors by the subject or time taken for the subject to make a predetermined number of errors may also be determined.

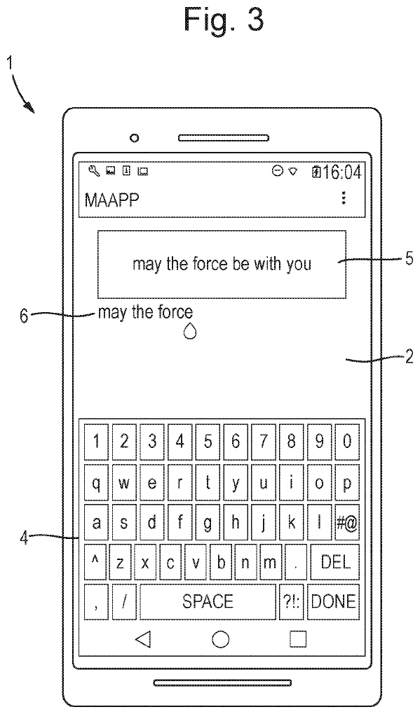

Another example of a specific task according to the present disclosure includes typing a character string. As shown in FIG. 3, the subject may be presented with a keyboard 4 to type on. This is preferably in the form of a touch keyboard 4 as conventionally used for a device 1 having a touch screen 2. As shown in FIG. 3, the subject may be instructed to type a specific phrase. The phrase may be presented visually in a first portion 5 of the screen 2 and the typed characters may be presented visually in a second portion 6 of the screen 2.

The keystrokes made by the user and timings thereof may be recorded by the device. Parameters relating to speed determined from the recorded data may include a rate of touch interactions, based on the time between successive touches. Variations in the rate of the touch interactions over time may be determined. This data may be recorded for each key pairing (i.e. successive typed characters) to reflect that different key pairings are more difficult to type than others. The time taken for the subject to complete the task may also be determined. Parameters relating to accuracy determined from the recorded data may include: the total number of errors by the subject, the rate of errors made by the subject, and/or the time for the subject to make a predetermined number of errors. An error may include typing an incorrect character and/or having to correct an action, such as using the delete key.

The above described tasks are examples of active testing. However, the same principles can be used for passive testing. For example, some applications on devices such as smart phones, tablet computers or smart watches require the user to touch specific target or type using a keyboard. Methods of the present disclosure may run in the background when the user used such applications and can obtain the same information. For example, data may be recorded during use of instant messaging or email applications which require typing.

Tests of a second type according to the present disclosure may include tasks requiring the subject to manipulate the device. Movement of the device may be recorded using one or more sensors of the device, e.g. gyros and/or accelerometers. Tests of the second type may be configured to test relatively gross sensory-motor function, including the subject's ability to walk or maintain a specific posture.

From a clinical standpoint, important characteristics of the subject's gross movement include strength, stability, the consistency of movement and the effects of fatigue on these characteristics, and differences between the left and right sides of the body. These characteristics can provide information regarding the subject's sensory-motor function. Therefore, methods according to the present disclosure include determining one or more parameters from the recorded touch interactions. These parameters may include indicators of strength and stability.

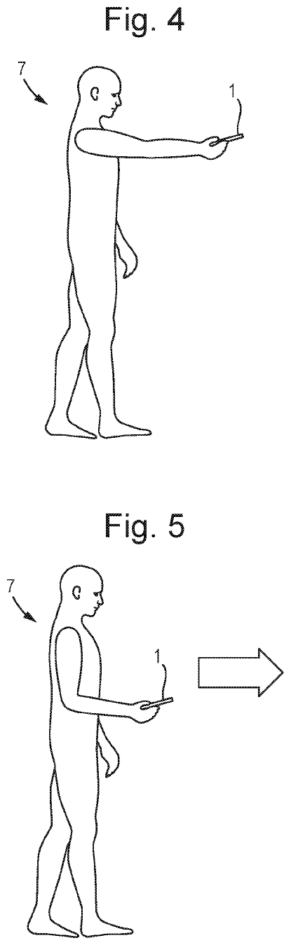

One example of a specific task of the second type according to the present disclosure includes holding the device in a predefined way for a predefined duration. As shown in FIG. 4, the subject 7 may be instructed to hold the device 1 in their hand (e.g. palm up), with their arm outstretched in front of them, for a set time period (e.g. 10 seconds). Such a test can also be performed by a smart watch or similar device worn on the wrist. The task may be performed for each hand separately.

Time series accelerometer data and gyro data may be recorded. This may provide data relating to each of the xyz axes, i.e. acceleration in along the axes and rotation about the axes. Parameters determined from the recorded data may include positional deviation from a predetermined position over time, i.e. how far the hand has moved from the starting position. Rotational deviation from a start position over time may be determined, i.e. whether there is a rotation in the arm or wrist, usually internally. Oscillation frequency, variation in oscillation frequency over time, oscillation magnitude, and variation in oscillation magnitude over time, may be determined i.e. how the subject's hand tremors.

Another example of a specific task of the second type according to the present disclosure includes moving the device in a predefined way. Such a task may include the subject walking with the device. As shown in FIG. 5, the subject 7 may be instructed to hold the device in their hand 1 (e.g. in their right palm and with their elbow held against their waist, extend their forearm out in front of them with the phone upright) and walk (e.g. for 60 seconds).

Time series accelerometer data and gyro data may be recorded. This may provide data relating to each of the xyz axes, i.e. acceleration in along the axes and rotation about the axes. From this data, the gait of the subject may be mapped in 3 dimensional space. Parameters determined from the recorded data may include stride frequency, variation in stride frequency over time, stride length, variation in stride length over time, stride width (e.g. with a wide or narrow stance), and variation in stride width over time. Whether the deice is dropped or the subject falls may also be determined.

The above described tasks are examples of active testing. However, the same principles can be used for passive testing. For example, the subject's gait can be analysed while walking with a device in their pocket or in their hand. Methods of the present disclosure may run in the background in the device. This may be triggered by specific activity signatures detected by the device (e.g. as provided by opensource activity API), or specific time intervals, for example.

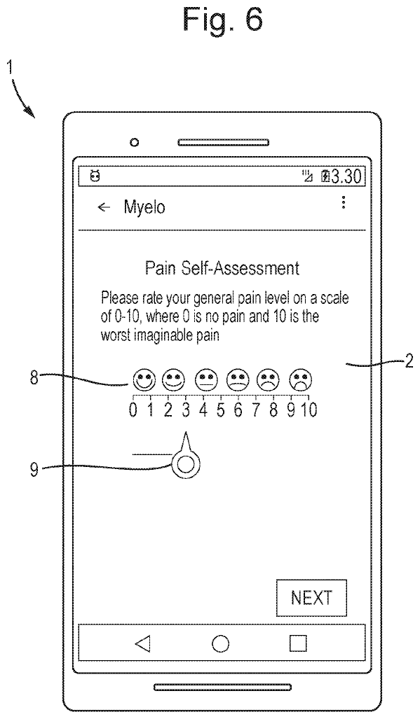

It should be readily understood that combinations of the above described tasks may be used to assess a subject. These may additionally be combined with questionnaires (Electronic Patient Reported Outcome Measures—ePROMS) delivered and recorded via the device. The questionnaire may be used to obtain information about the subject's condition including levels of pain, ability to perform certain everyday tasks. As shown in FIG. 6, the subject may be asked to rate their current pain level, e.g. on a visual scale 8, using a slider 9 by interacting with the touch screen 2 of the device 1.

An assessment of neuromuscular disease in the subject can be provided by comparing the determined parameters relating to the various tasks with predetermined parameters. The comparison may assess the severity of neuromuscular disease in the subject and or the trajectory, e.g. improvement or degeneration of the disease. The predetermined parameters may include a baseline for the subject. This baseline may be determined from previously obtained data for the subject when performing the same tasks. The predetermined parameters may also include a baseline for a predetermined population group, e.g. male/female, age groups. The predetermined parameters may also include a baseline for a predetermined clinical group, such as those with a specific type of injury or illness.

An assessment score may be determined based on the comparison between the determined parameters and the predetermined parameters, wherein the assessment score indicates the severity of neurological disease in the subject. The outcome of comparisons for the various tasks and various parameters may be aggregated to provide a single score. Weightings for each may be determined based on the specific disease, for example.

Determination of parameters, and comparisons with predetermined parameters may be performed by one or more processors. The one or more processors may be processors of the device or a remote device or server, as described above.

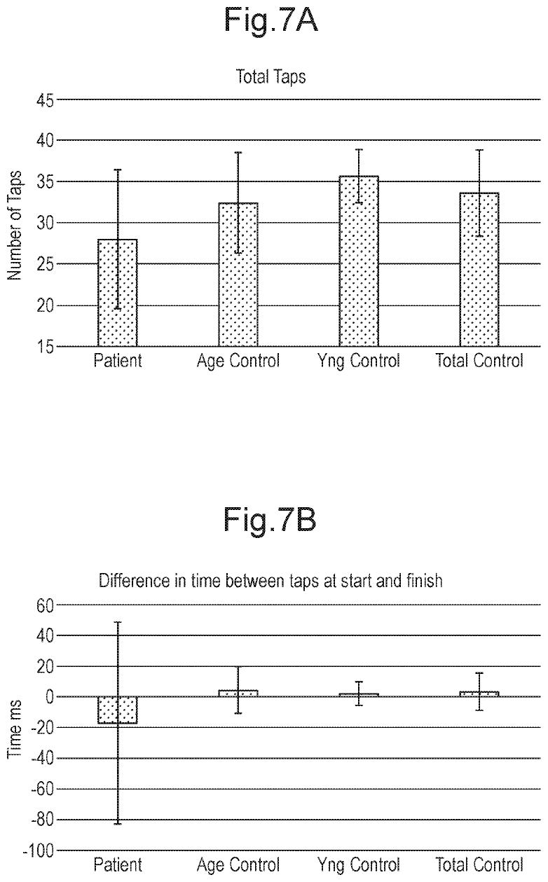

FIGS. 7 to 9 show some examples of data obtained using some of the tests described above.

FIG. 7A is a chart showing the average total number of taps for the test described with reference to FIG. 1. It can be seen that the number of taps for the patient group, suffering DCM, is significantly lower than for an age controlled group, a younger control group and a total control group. FIG. 7B is chart showing the difference in time between taps at the start of the test and the end of the test. It can be seen that the patient group got slower, whereas the control groups got faster over the duration of the test.

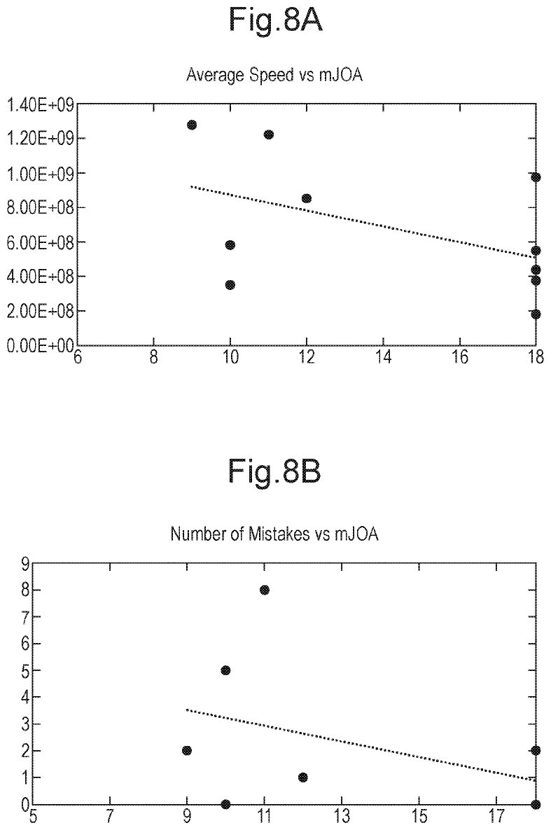

FIG. 8A is a chart showing the average typing speed of subjects during the test described with reference to FIG. 3 vs their mJOA score, an indicator of DCM severity such that a lower mJOA score indicates a higher DCM severity (i.e. a score of 18 is normal, and a score of 0 the most severe form of disease). It can be seen that subjects with lower mJOA scores typed more slowly on average than those with higher mJOA scores. FIG. 8B is a chart showing the number of mistakes made against mJOA score. It can be seen that subjects with lower mJOA scores made more mistakes on average than those with higher mJOA scores.

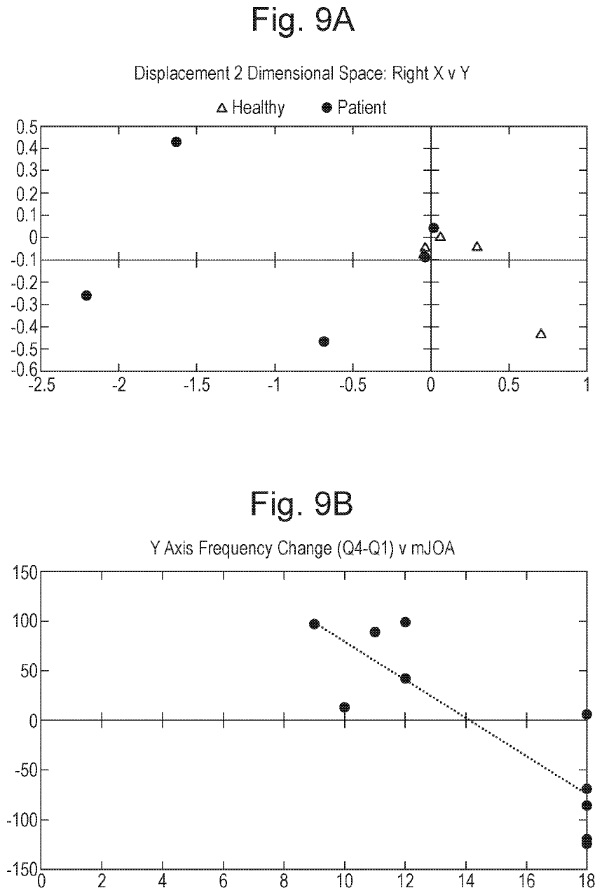

FIG. 9A is a chart showing the two-dimensional, positional displacement of the device during the test described in relation to FIG. 4, for a healthy group and a patient group suffering DCM. It can be seen that larger displacement, on average, is found in the patient group. FIG. 9B shows the difference in average oscillation frequency in the y-axis (up/down directions) between fourth (Q4) and first quartiles (Q1) of the test against mJOA score. It can be seen that oscillation frequency increases between Q1 and Q4 for those with lower mJOA scores but decreased for those with higher mJOA scores. This indicates that people with DCM gradually lose their ability to maintain stability with the task, whereas health individuals can reinforce their stability.

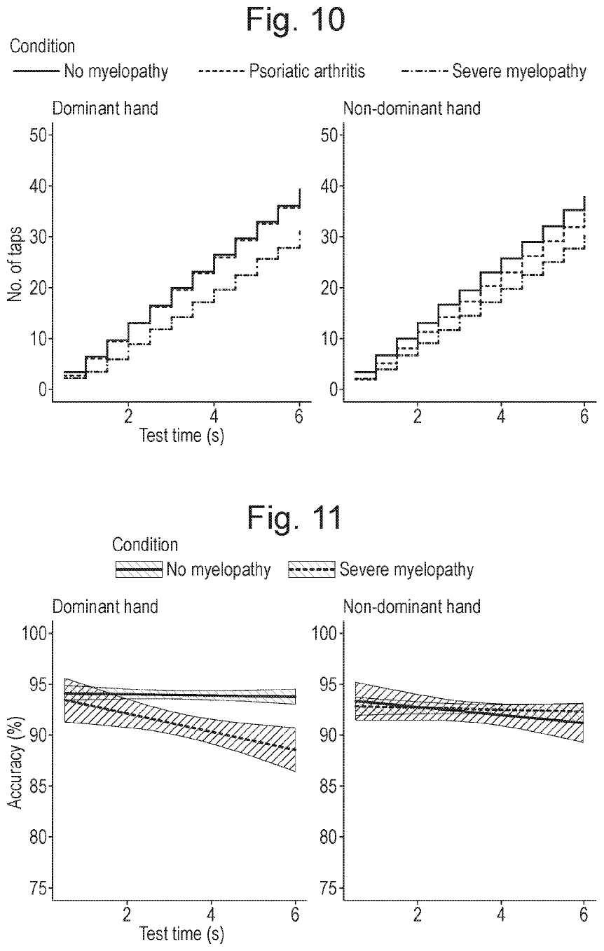

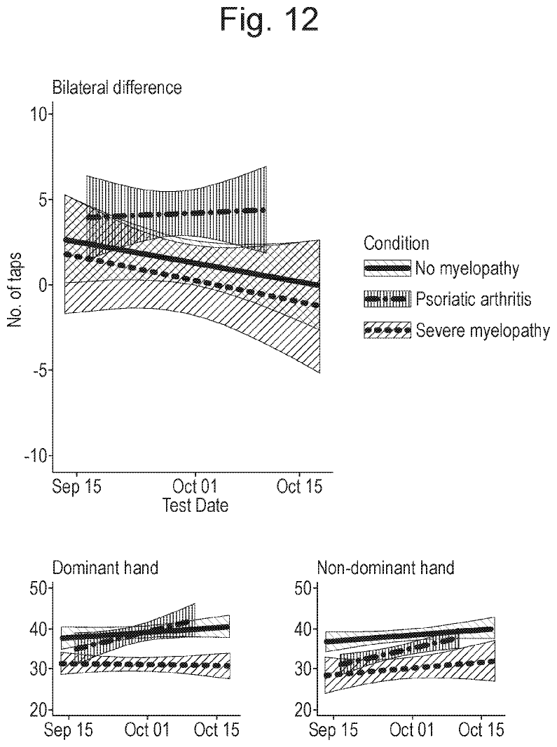

FIGS. 10 to 13 show further data from a test as described in relation to FIG. 1, namely tapping a target. FIG. 10 shows the number of taps over the duration of the test for dominant and non-dominant hands for groups with no myelopathy, psoriatic arthritis and severe myelopathy. It can be seen that subjects with severe myelopathy tap slower with both dominant and non-dominant hands during the tests. FIG. 11 shows tapping accuracy over the duration of the test for dominant and non-dominant hands for groups with no myelopathy and severe myelopathy. It can be seen that subjects with severe myelopathy show a decline in accuracy over the duration of the test and this is more pronounced in the dominant hand. FIG. 12 shows bilateral difference in the number of taps over several weeks of testing. It can be seen that subjects with psoriatic arthritis exhibit unilateral dominance (top panel) over time, and that subjects with severe myelopathy continue to tap slower with both dominant and non-dominant hands (bottom panels).

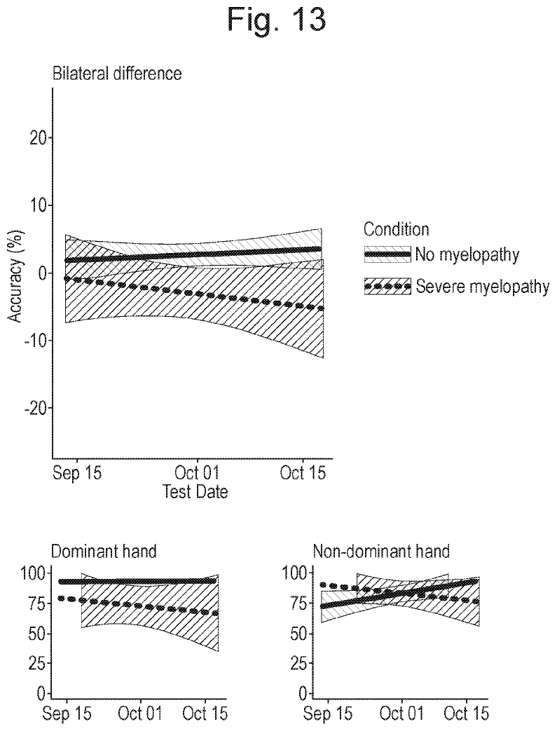

FIG. 13 shows bilateral difference in tapping accuracy over several weeks of testing. It can be seen that accuracy continues to decline over time in subjects with severe myelopathy (bottom panels), and that this decline exhibits unilateral dominance in subjects with severe myelopathy (top panel).

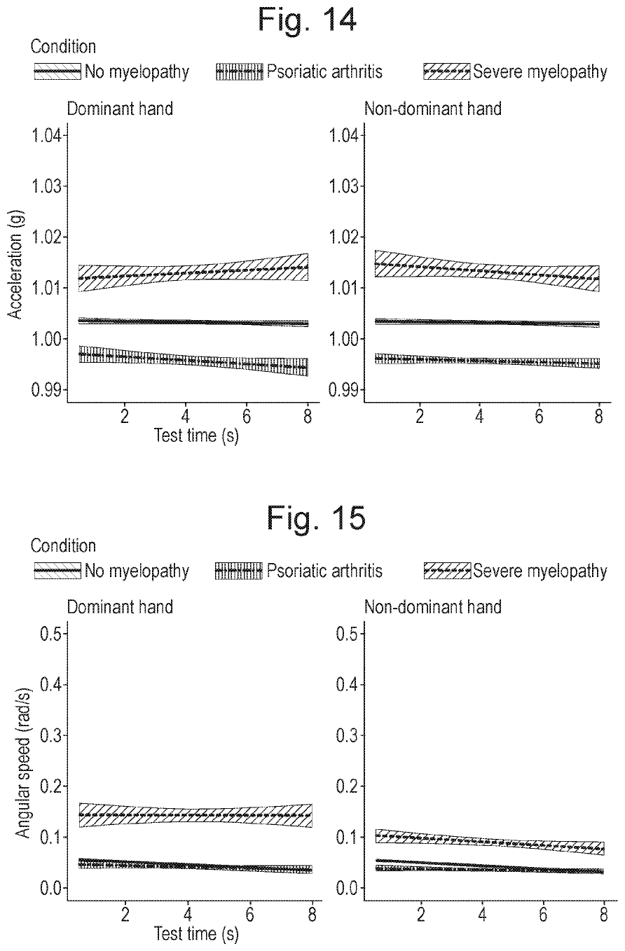

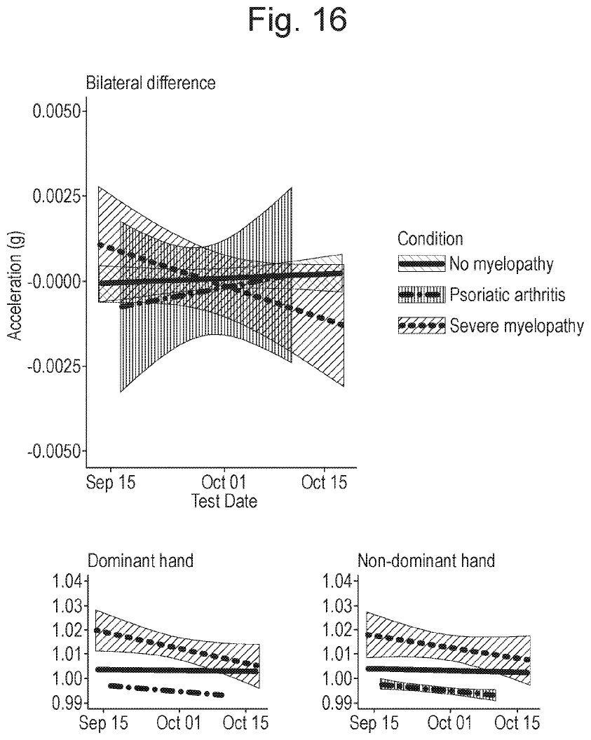

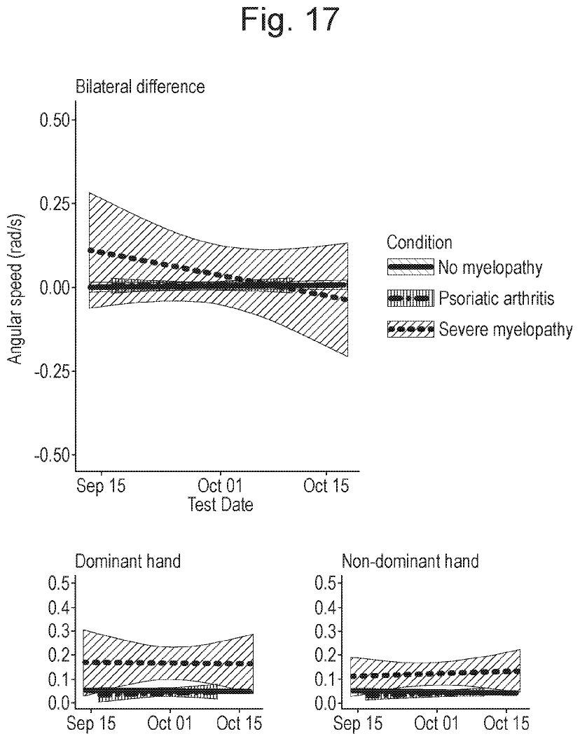

FIGS. 14 to 17 show further data from a test as described in relation to FIG. 4, i.e. holding a device. FIG. 14 shows acceleration for dominant and non-dominant hands for groups with no myelopathy, psoriatic arthritis and severe myelopathy. FIG. 15 shows angular speed for dominant and non-dominant hands for groups with no myelopathy, psoriatic arthritis and severe myelopathy. It can be seen from these data that subjects with severe myelopathy hold the device less steadily during the tests, but that subjects with psoriatic arthritis hold it more stiffly. FIGS. 16 and 17 respectively show bilateral differences for acceleration and angular speed over several weeks of testing. It can be seen from these data that subjects with severe myelopathy continue to hold the device less steadily over time (bottom panels) and that they inconsistently exhibit unilateral dominance (top panels). Similarly, subjects with psoriatic arthritis continue to hold the device more stiffly over time (bottom panels) and they inconsistently exhibit unilateral dominance (top panels). This data supports the observation that myelopathy is a neurological disease, so affects the consistent action of the muscle, whereas arthritis causes a stiffening of the joints.

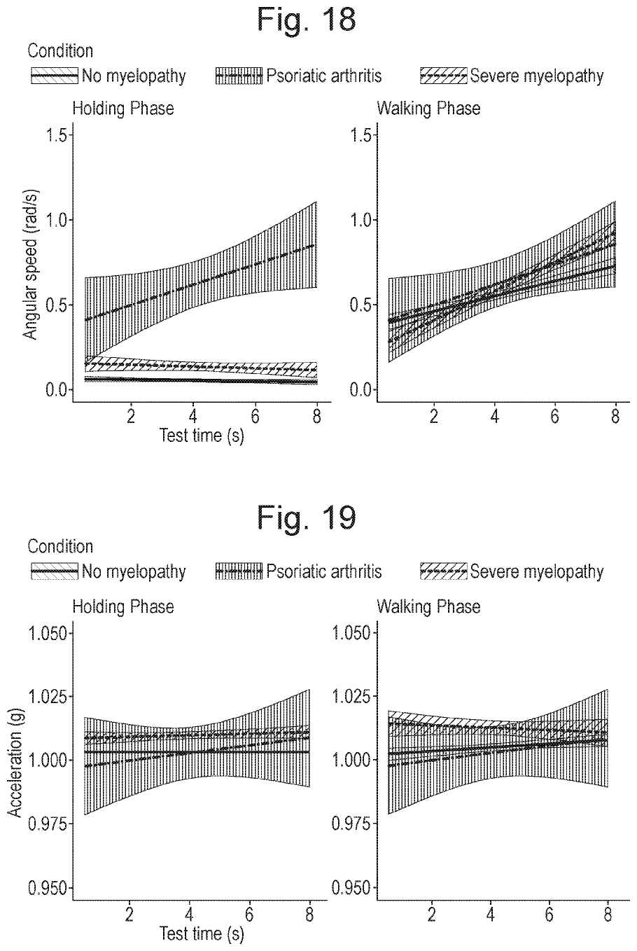

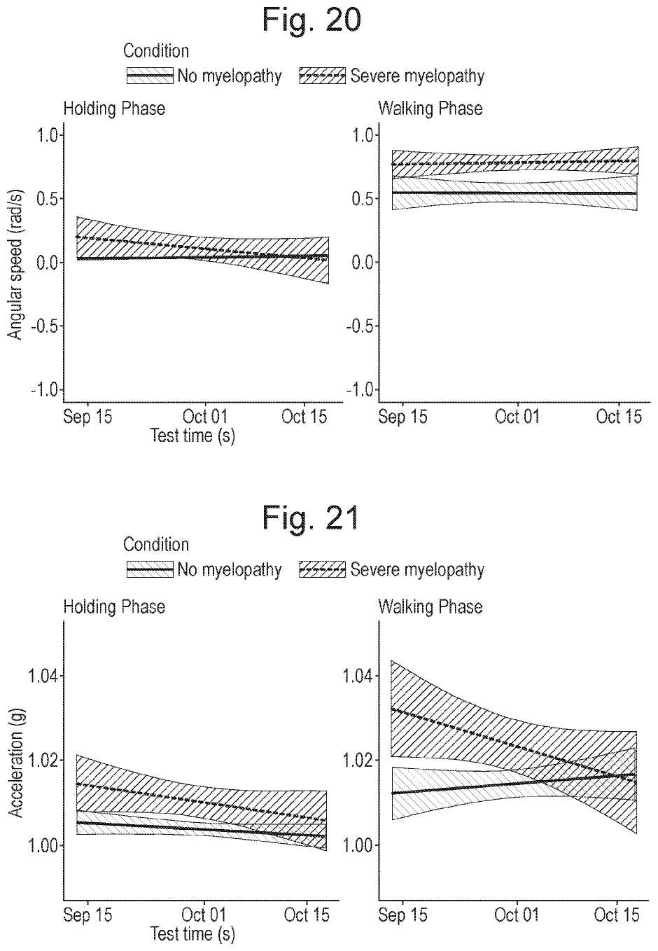

FIGS. 18 to 21 show further data from a test as described in relation to FIG. 5, i.e. walking with a device, but with an initial holding phase without walking. FIG. 18 shows angular speed for the holding phase and the walking phase in groups with no myelopathy, psoriatic arthritis and severe myelopathy. FIG. 19 shows acceleration for the holding phase and the walking phase in groups with no myelopathy, psoriatic arthritis and severe myelopathy. It can be seen from these data that, during the tests, subjects with psoriatic arthritis and severe myelopathy hold the device less steadily during both the holding phase without walking, and the walking phase. FIGS. 20 and 21 respectively show angular speed and acceleration over several weeks of testing. It can be seen from this data that, over several weeks of testing, subjects with severe myelopathy continue to hold the device less steadily during both the holding phase without walking, and the walking phase.

1. A method for assessing neuromuscular disease in a subject using a handheld or wearable computing device, the method comprising:
recording the subject performing a plurality of tasks on a device, wherein at least one of the plurality of tasks requires the subject to interact with the device by touching the device, including recording touch interactions with the device using one or more sensors of the device, and wherein at least one of the plurality of tasks requires the subject to manipulate the device, including recording movement of the device using one or more sensors of the device;
wherein at least one task of the plurality of tasks comprises background monitoring of the device, obtained via sensor outputs not resulting directly from performance of a task presented to the subject via a user interface of the device specifically for assessing neuromuscular disease;
determining one or more parameters from the recorded touch interactions and the recorded background monitoring of the computing device; and
comparing the determined parameters for each task with predetermined parameters.

2. The method of claim 1, wherein the plurality of tasks includes a task presented to the subject via a user interface of the device.

3. (canceled)

4. The method of claim 1, wherein at least one of the plurality of tasks comprises repeatedly touching a single target.

5. (canceled)

6. The method of claim 1, wherein one of the plurality of tasks comprises typing a character string.

7. The method of claim 1, wherein the one or more parameters include indicators of interaction speed and/or accuracy.

8. The method of claim 1, wherein for a task requiring the subject to touch the device the one or more parameters include one or more of: a rate of touch interactions, variation in the rate of the touch interactions over time, and time taken for the subject to complete the task.

9. The method of claim 1, wherein for a task requiring the subject to touch the device the one or more parameters include indicators of interaction accuracy.

10. The method of claim 1, wherein the one or more parameters include one or more of:
accuracy of the touch interactions, variation in the accuracy of the touch interactions over time, total number of errors by the subject, rate of errors by the subject, time for the subject to make a predetermined number of errors.

11. (canceled)

12. (canceled)

13. (canceled)

14. The method of claim 11, wherein one of the plurality of tasks includes holding the device in a predefined way for a predefined duration.

15. The method of claim 11, wherein for a task requiring the subject to manipulate the device the one or more parameters include one or more of: positional deviation from a predetermined position over time, rotational deviation from a predetermined orientation over time, oscillation frequency, variation in oscillation frequency over time, oscillation magnitude, and variation in oscillation magnitude over time.

16. The method of claim 11, wherein one of the plurality of tasks comprises moving the device in a predefined way.

17. The method of claim 1, wherein one of the plurality of tasks comprises walking with the device, wherein the one or more parameters include one or more of: stride frequency, variation in stride frequency over time, stride length, variation in stride length over time, stride width, variation in stride over time.

18. (canceled)

19. (canceled)

20. The method of claim 1, wherein one or more of the plurality of tests is performed bilaterally; and the one or more determined parameters includes differences between parameters for each side.

21. The method of claim 1, wherein the predetermined parameters include one or more of: a baseline for the subject, a baseline for a predetermined population group, a baseline for a predetermined clinical group.

22. The method of claim 1, comprising determining an assessment score based on the comparison between the determined parameters and the predetermined parameters, wherein the assessment score indicates the severity and progression of neurological disease symptoms in the subject.

23. The method of claim 1, wherein the neuromuscular disease is a degenerative neuromuscular disorder comprising deterioration of dexterity.

24. (canceled)

25. A computer program that when executed on a handheld or wearable computing device configured to perform the following steps:
enabling the subject to perform a plurality of tasks on a device, wherein at least one of the plurality of tasks requires the subject to interact with the device by touching the device, including recording touch interactions with the device using one or more sensors of the device, and wherein at least one of the plurality of tasks requires the subject to manipulate the device, including recording movement of the device using one or more sensors of the device;
wherein at least one of the plurality of tasks comprises background monitoring of the device, during which sensor outputs are gathered not as a result of performance of any task presented to the subject via a user interface of the device specifically for assessing neuromuscular disease;
determining one or more parameters from the recorded touch interactions and the recorded movement of the computing device; and
comparing the determined parameters for each task with predetermined parameters.

26. A system for assessing neuromuscular disease in a subject, the system comprising:
a device comprising a touch screen and one or more sensors, wherein the touch screen is configured to present a plurality of tasks to the subject and record the subject performing the plurality of tasks, wherein cat least one of the plurality of tasks requires the subject to interact with the device by touching the device, and the one or more sensors are configured to record touch interactions with the device, and wherein at least one of the plurality of tasks requires the subject to manipulate the device, and the one or more sensors are configured to record movement of the device;
wherein the plurality of tasks includes a task performed during alternative use of the device, not a task presented to the subject via a user interface of the device specifically for assessing neuromuscular disease;
and
one or more processors configured to determine one or more parameters from the recorded touch interactions and from the recorded movement of the device and to compare the determined parameters with predetermined parameters.

27. (canceled)

28. The system of claim 26, wherein the one or more processors is configured to determine an assessment score based on the comparison between the determined parameters and the predetermined parameters, wherein the assessment score indicates the severity of neurological disease in the subject.

29. The system of claim 26, wherein the assessment score is determined by applying different weightings to the determined parameters and the predetermined parameters based on the neurological disease.
